(19) United States
(12) Patent Application Publication
(10) Pub. No.: US 2010/0319079 A1
(43) Pub. Date: Dec. 16, 2010

(54) ISOLATED PROLIFERATING CELLS WITH STEM CELL PROPERTIES FROM ADULT TISSUE OF POIKILOTHERMIC VERTEBRATES, STABLE CELL CULTURES THEREOF, AND METHODS FOR THEIR PREPARATION

(75) Inventors: Charli Kruse, Herrnburg (DE); Emel Singh, Luebeck (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: Fraunhofer-Gesellschaft zur Foerderung der angewandten Forschung e.V., Muenchen (DE)

(21) Appl. No.: 12/664,916

(22) PCT Filed: Feb. 27, 2008
(86) PCT No.: PCT/EP2008/001550

§ 371 Date: Aug. 12, 2010

(30) Foreign Application Priority Data
Jun. 27, 2007 (DE) 10 2007 029 699.3

(51) Int. Cl. A01K 67/00; C12N 5/071; C12N 9/00; C12P 7/64; G01N 33/53

(52) U.S. Cl. 800/21; 435/379; 435/325; 435/183; 435/134; 435/7.21

(57) ABSTRACT

A method of preparing adult proliferating cells with stem cell properties includes removing tissue from pronephros or pyloric appendates of an intestine of poikilothermic vertebrates, comminuting removed tissue, cultivating comminuted tissue, and propagating cells persisting in a culture.

### RELATED APPLICATIONS

This is a §371 of International Application No. PCT/EP2008/001550, with an international filing date of Feb. 27, 2008 (WO 2009/000347 A1, published Dec. 31, 2008), which is based on German Patent Application No. 10 2007 029 699.3, filed Jun. 27, 2007, the subject matter of which is incorporated by reference.

### TECHNICAL FIELD

This disclosure relates to a method for the preparation of isolated proliferating cells with stem cell properties and to the corresponding stable cell cultures from adult tissue of poikilothermic animals, in particular fish (Pisces), and to the cells and cell cultures prepared therewith.

### BACKGROUND

The designation “stem cells” is used for those cells which are capable to divide indefinitely and can differentiate into different types of cells under suitable circumstances or by means of suitable stimuli. Stem cells have the potential to develop into cells having a characteristic shape and specialized functions. It is often only possible to a very limited extent or not at all to cultivate these specialized cells outside of the organism such that they have to be prepared indirectly by means of cultivating stem cells.

Hitherto, it has only been possible to isolate and investigate embryonic stem cells from a few fish species (zebrafish and medaka) [Ma et al., 2001, Proc. Natl. Acad. Sci. U.S.A. 98:2461-2466; Epub 2001, February 13; Hong et al., 1996, Mech. Develop., 60:33-44], whereas only very few studies of adult stem cells from fish are known [Tawk and Vriz 2003, Med. Sci. (Paris) 19:465-471; Alvaro and Tsonis et al., 2006, Nat. Rev. Genet., 7:873-884]. However, these studies were performed on complex fish tissues, typically in vivo on the fin, heart and brain, and did not allow for the recovery of isolated stem cells or stable stem cell cultures.

Isolation and cultivation of haematopoietic stem cells from kidney tissue of carps is described in Developmental and Comparative Immunology, Vol. 31, No. 7, 696-707 (2007). The obtained stem cells are not capable of differentiating into cells of different germ layers. The pronephros is also mentioned as a starting material unsuitable for the isolation of the respective stem cells due to the extremely low occurrence (close to 0%) (Abstract and page 703).

It could therefore be helpful to recover isolated proliferating cells with stem cell properties, in particular unlimited divisibility and multipotency or pluripotency, from adult tissue of poikilothermic vertebrates, in particular fish, and to establish stable proliferating cell cultures thereof, by means of which a wide spectrum of different differentiated cells can be produced.

### SUMMARY

We provide a method of preparing adult proliferating cells with stem cell properties, including removing tissue from pronephros or pyloric appendages of an intestine of poikilothermic vertebrates, comminuting removed tissue, cultivating comminuted tissue, and propagating cells persisting in a culture.

We also provide proliferating cells with stem cell properties, obtained by the method from the pronephros tissue of a fish, wherein the cells have unlimited divisibility and differentiate into cells of at least both of the ectodermal and mesodermal germ layers.

We further provide a cell culture including the cells in a culture medium which permits stable maintenance and propagation of the cells, substantially without differentiation.

We also further provide a differentiation culture including the cells and cells differentiated therefrom in a differentiation medium, wherein the differentiation medium includes no additional growth or differentiation factors.

We still further provide differentiated cells obtained from the cells that develop into tissue-like or organ-like multicellular systems in vitro.

We also further provide a method of screening pharmaceutical drugs including providing the cells, contacting the cells with a test agent, and evaluating effect(s), if any, on the cells.

We also provide a method of producing fish oils or fatty acids in vitro including contacting the cells with enzymes with an optimum activity at low temperatures, and recovering the fish oils or fatty acids.

We further provide a method of breeding fish including reproductive cloning of poikilothermic vertebrates obtained from the cells.

We still further provide a method of implanting foreign gene material or foreign matter including introducing the foreign gene material or foreign matter into the cells.

We also further provide a kit including the cells, and expedients and/or reagents.

### BRIEF DESCRIPTION OF THE DRAWINGS

FIG. 1 shows a schematic depiction of the workflow of the method, starting from the pronephros of a fish.

FIG. 2 shows the immunohistological detection of differentiated cells (muscle cells) having mesodermal properties prepared in accordance with our method from sturgeons. The cells were stained with antibodies to α-smooth-muscle actin (SMA). The nuclei were additionally stained with DAPI.

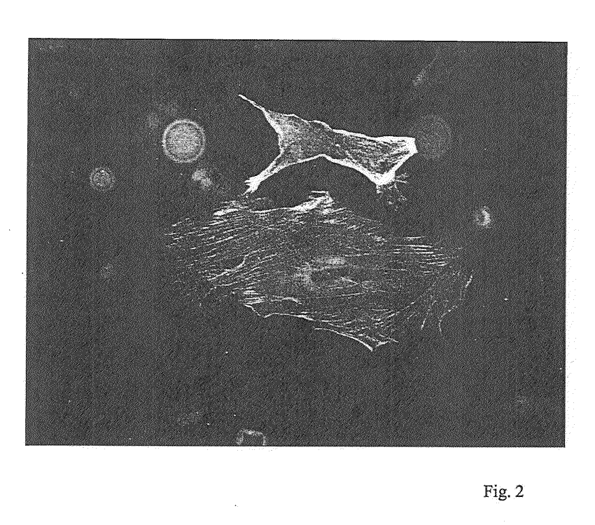

FIG. 3 shows the immunohistological detection of neuroglia cells having ectodermal properties prepared in accordance with our method from sturgeons. The cells were stained with antibodies to glial fibrillary acidic protein (GFAP). The nuclei were additionally stained with DAPI.

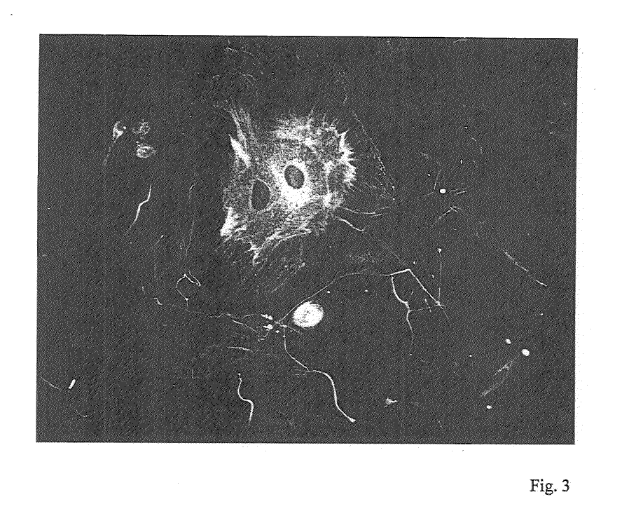

FIG. 4 shows the immunohistological detection of differentiated cells having structural proteins (cytokeratin) and the translation marker vigilin which have been prepared in accordance with our method from sturgeons. The cells were stained with antibodies to pancytokeratin (red) and vigilin (green). The nuclei were additionally stained with DAPI.

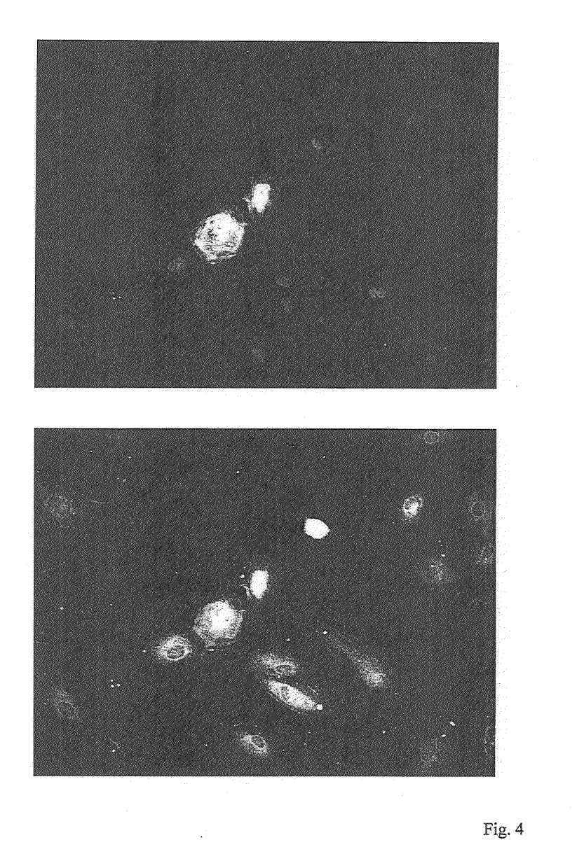

### DETAILED DESCRIPTION

The poikilothermic vertebrate may be a fish, amphibian or reptile. It is preferably a fish, in particular, a commercial fish. A few non-limiting examples of suitable species are sturgeon, herring, trout, salmon, eel, carp.

Pronephros tissue is preferably used as the adult starting tissue. Possible alternatives include pancreas, intestine, pyloric appendages of the intestine, male or female gonads, for example. In the case of fish, the pronephros which has haematopoietic functions in fish is preferably used. It is known that the haematopoietic stem cells of mammals detach from the surface under cell culture conditions after differentiating into mature blood cells. In contrast, the cells from fish pronephros do not detach, still adhered to the surface after more than 18 passages and have characteristics of stem cells, similar to those isolated from exocrine glands.

The proliferating cells provided have stem cell properties, i.e., they exhibit unlimited divisibility and are furthermore multipotent or pluripotent, i.e., they can spontaneously differentiate into cells of at least two germ layers (ectodermal and mesodermal).

It is extremely surprising that our simple method allows for the preparation of adult stem cells which reflect different germ layers in their differentiation potential and thus can at least be called multipotent. The stem cells produced by our method are easily recovered and spontaneously differentiate into different types of cells in a reproducible manner. Hitherto, muscle cells, neuroglia cells, epithelial and endothelial cells have been verified; however, the available types of cells are certainly not limited to these and findings already exist which point to further types of cells.

A feeder layer is neither required for the cultivation of the stem cells nor for the differentiation, and the cells also do not have to be transplanted to differentiate. The term “feeder cells,” as used herein, encompasses all the cells which promote the growth of the actual cells to be cultivated by releasing growth factors and/or providing an extracellular matrix or preventing the differentiation of the stem cell culture.

It is also remarkable that the stem cells can proliferate in an adhesion culture. In contrast, conventional haematopoietic stem cells are typically grown in a suspension culture.

The cells in our stem cell cultures have maintained their capability to self-renew and divide unlimitedly after so far more than 25 passages over 6 months and the cultures are still stable.

Furthermore, the obtained stem cells can be stored frozen (preferably in liquid nitrogen) and retain their differentiability and vitality without any changes.

The cells and cell cultures can be cultivated at temperatures lower than 37° C. (typical for mammalian cell cultures). The appropriate temperature range depends on the type and origin of the fish species used as the tissue source and thus can encompass a wide spectrum of from about 1-35° C. For tropical fish used as the starting organism, an adequate cultivation temperature may be in the upper range, e.g., about 25-35° C., whereas it can be 1-10° C. for cells from fish from colder regions, for example. In particular for fish from the temperate zone, the temperature range will be typically within a range of from 5-27° C., preferably 10-25° C., more preferably 17-24° C., in particular 18-20° C. or approximately room temperature. One skilled in the art can easily determine the appropriate or optimal temperature range in each case by routine experimentation.

The stem cells and stem cell cultures have a wide spectrum of applications.

An example is the provision of complex cellular test systems, e.g., to evaluate cellular interactions or to analyze the effect of different noxa, chemicals, in particular, potential pharmaceutical drugs, or cosmetics as well.

These cellular systems preferably comprise several types of cells, e.g., those also occurring side by side in natural tissues such that they can also be tested simultaneously.

The stem cells and stem cell cultures may also be used to develop tissue-like or organ-like multicellular systems, preferably from several types of cells, in vitro.

Additionally, these can be used for food and feed production. For example, they may be used to prepare fish-specific biomass, such as, for example, artificial meat, fish meal, fish oil or individual components of these cells, such as, for example, omega fatty acids or proteins with an amino acid composition typical for fish.

A more general application of these cells, cell cultures and multicellular systems is the use as an in vitro system to produce desired substances in vitro.

These substances may be specific substances which are naturally produced in the starting organisms, e.g., fish. Specific examples are certain proteins, e.g., cold-adapted enzymes with an activity optimum at lower temperatures (e.g., about 10° C.), with possible applications in food ripening, among other things. Such enzymes could be produced by secretory cells, for example, which are formed by spontaneous or targeted differentiation from the stem cells.

The stem cells and cell cultures can also be used as recipient cells or vehicles to receive foreign gene material or other foreign matter according to known methods. The expression of the foreign gene material then permits production of heterogeneous proteins in these cells and cultures.

A particular advantage of such cells and cell cultures is that they already grow well at lower temperatures than 37° C. (typical for mammalian cell cultures), e.g., within a range of from 1-35° C., preferably 5-27° C. or 17-24° C., in particular 18-20° C. or approximately room temperature, making an energy-intensive heating unnecessary.

Yet another application of the cells is the reproductive cloning of poikilothermic vertebrates, in particular for fish breeding (cloned fishes).

We also provide a kit comprising the stem cells, and further expedients or reagents, e.g., reagents for cultivating the cells or diagnostic reagents.

The structural cytokeratin proteins are primarily formed in epithelial and endothelial cells. The protein vigilin is typical for protein-synthesizing cells and was hitherto found in all the stem cells.

Besides these detections, it was also possible to demonstrate that cells are present which store fat droplets, and that a plurality of different morphologies of the cells can be identified which suggests a wider variety of cells (data not shown).

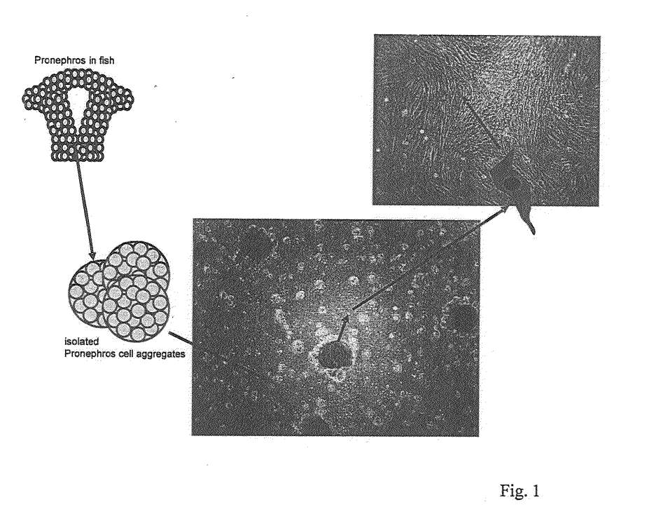

Corresponding to the diagram shown in FIG. 1, pronephros tissue mechanically and enzymatically comminuted is cultivated to recover the cells. In this process, no complete tissue blocks are cultivated but intact cell aggregates smaller than ca. 200 μm which are placed in culture vessels in which the desired cells subsequently grow or form anew.

These cells and cell aggregates are cultivated in culture vessels for several weeks. The medium is replaced every 2-3 days, removing all the differentiated cells. The cells persisting in culture are undifferentiated cells with unlimited divisibility.

It should be noted that, on the one hand, the enzymatic tissue digestion takes place at 37° C. which the cells seem to tolerate well, and that, on the other hand, the cells grow very well at 18-20° C. whereas the starting animals themselves often thrive ideally at lower temperatures.

Such primary cells with a very high multiplication capacity from pronephros of fish have not been described yet. Hitherto, these cells demonstrated unlimited divisibility and could be held in culture continuously for ca. 6 months.

The following non-limiting example is intended to describe preferably applied steps of the method in more detail.

The general instructions, as are customary for methods for the cultivation of animal cells and in particular mammalian cells, should be observed. A sterile environment in which the method is to be performed is to be maintained in any case—even if no further description is given.

The following buffers and media are provided:

| | |
|---|---|
| HEPES stock solution | 2.383 g of HEPES to 100 ml of A. bidest. |
| (pH 7.6) | |
| HEPES/Eagle's Medium | 90 ml of Modified Eagle's Medium (MEM) |
| | 10 ml of HEPES stock solution |
| Isolation medium | 32 ml of HEPES/Eagle's Medium |
| (pH 7.4) | 8 ml of 5% BSA in A. bidest. |
| | 200 μl of 0.1M CaCl₂ |
| | 100 μl of trasylol (200,000 KIU) |
| Digestion medium | 20 ml of isolation medium |
| (pH 7.4) | 4 mg of collagenase |
| Incubation medium | Dulbecco's Modified Eagle's Medium |
| | (DMEM) |
| Differentiation medium | 380 ml of DMEM |
| | 100 ml of FCS inactivated at 54° C. for 30 min |
| | (or fungi or algae extract) |
| | 5 ml of glutamine (PAA Laboratories GmbH) |
| | 5 ml of (3.5 μl of β-mercaptoethanol to 5 ml |
| | of PBS) |
| | 5 ml of non-essential amino acids |
| | (GIBCO BRL) |
| | 5 ml of penicillin/streptomycin (PAA) |

### Example 1

### 1. Preparation of Tissue and Isolation of Cells

The pronephros of a sturgeon is removed from the surface of the upper body cavity, starting directly behind the skull. The tissue is then comminuted in a beaker with digestion medium using fine scissors. The suspension is subsequently gassed for 1 min with carbogen and incubated for 20 min at 37° C. in a shaker at 200 cycles/min. Thereafter, the medium is carefully aspirated and the tissue pieces are in each case washed twice with 10 ml of isolation medium and 5-10 ml of digestion medium are once more added to the tissue.

After another gassing with carbogen for 1 min and incubation for 15 min at 37° C. in a shaker at 200 cycles/min, the tissue pieces are comminuted by successively drawing them into a 10 ml, 5 ml and 2 ml glass pipette and are pressed through a single-layer filter fabric having a mesh size of ca. 200 p.m. The cells thus singularized are now washed once to twice in incubation medium (37° C.) and are each time centrifuged for 5 min at 90 g (800 rpm). The at last obtained pellet is resuspended in incubation medium and distributed into tissue culture dishes (25 cm²).

### 2. Cultivation of Cells

The tissue culture dishes with the isolated cells are cultivated in an incubator at 18-20° C. and 5% CO₂. The medium is replaced every 3-4 days. In the process, all the non-adhering and many differentiated cells are removed.

Once the cells completely cover the bottom (are thus confluent)—for the first time roughly on the 10th day of the cultivation—the cells are passaged with a solution consisting of 2 ml of PBS, 1 ml of trypsin and 2 ml of incubation medium at 37° C. In the process, the cells detach from the bottom of the culture dish. The cell suspension is centrifuged for 5 minutes, the supernatant is aspirated and the cells are resuspended in 5 ml of incubation medium, transferred to a medium-sized cell culture bottle (75 cm²) and 10 ml of incubation medium are added. Replacement of the medium takes place every three days.

On the fourteenth day of the cultivation, the cells are once more passaged, but this time with 6 ml of PBS, 2 ml of trypsin and 6 ml of incubation medium. The cell suspension is centrifuged for 5 minutes, the supernatant is aspirated and the cells are resuspended in 15 ml of incubation medium, transferred to 3 medium-sized cell culture bottles and 10 ml of incubation medium are added in each case.

The cells are cultivated further and passaged and seeded as often as it takes until the cells achieve a semiconfluent to confluent state.

For the differentiation, the cells can be transferred to a differentiation medium (e.g., having the composition indicated in the table above) and differentiated under known cultivation conditions. Additional differentiation and growth factors are not necessarily required for the differentiation as the cells can spontaneously differentiate into different types of cells. Nonetheless, it can be advantageous to add such differentiation and growth factors to promote a targeted differentiation into certain types of cells. Such factors which specifically promote the differentiation of stem cells into certain types of cells are known for an entire range of types of cells and can be employed analogously to known protocols.

1. A method of preparing adult proliferating cells with stem cell properties, comprising:
removing tissue from pronephros or pyloric appendages of an intestine of poikilothermic vertebrates,
comminuting removed tissue,
cultivating comminuted tissue, and
propagating cells persisting in a culture.

2. The method according to claim 1, wherein the tissue is gently comminuted such that cell aggregates smaller than about 200 μm are substantially maintained in the resulting small tissue pieces, and
comminuted tissue is initially cultivated in tissue culture vessels, the majority of the differentiated cells dying quickly over several days and detaching from the stem cells, the stem cells subsequently adhering on bottom portions of the tissue culture vessels,
and remaining tissue and non-adhering differentiated cells are largely separated by a first medium replacement, and
remaining non-adhering cells are separated by further medium replacements within several days.

3. The method according to claim 1, wherein the poikilothermic vertebrate is a fish.

4. The method according to claim 1, wherein cultivating and propagating of the cells persisting in the culture takes place at 1-35° C.

5. The method according to claim 4, wherein cultivating and propagating of the cells persisting in the culture takes place at 5-27° C.

6. Proliferating cells with stem cell properties, obtained by the method according to claim 1 from the pronephros tissue of a fish, wherein the cells have unlimited divisibility and differentiate into cells of at least both of the ectodermal and mesodermal germ layers.

7. The cells according to claim 6, wherein the cells maintain their capability to self-renew and divide unlimitedly even after freezing/cryo-conservation and do not differentiate.

8. The cells according to claim 6, wherein the cells have the capability to proliferate in an adhesion culture.

9. A cell culture comprising the cells according to claim 6 in a culture medium which permits stable maintenance and propagation of the cells, substantially without differentiation.

10. The cell culture according to claim 9, which is an adhesion culture.

11. The cell culture according to claim 9, wherein the culture medium comprises no feeder layer.

12. The cell culture according to claim 9, wherein the cells retain their capability to self-renew and divide unlimitedly for more than 25 passages.

13. The cell culture according to claim 12, wherein the cells retain their capability to self-renew and divide unlimitedly for more than 50 passages.

14. The cell culture according to claim 9, wherein the culture medium has a temperature in the range of from 1-35° C.

15. A differentiation culture comprising the cells according to claim 6 and cells differentiated therefrom in a differentiation medium, wherein the differentiation medium comprises no additional growth or differentiation factors.

16. Differentiated cells obtained from the cells according to claim 6 that develop into tissue-like or organ-like multicellular systems in vitro.

17. The differentiated cells according to claim 16, wherein the multicellular systems comprise several different cells.

18. A method of testing a chemical comprising:
providing the cells according to claim 6;
contacting the cells with a test chemical; and
evaluating effect(s), if any, on the cells.

19. A method of producing proteins comprising enzymes with an optimum activity at low temperatures, fish oils or fatty acids comprising:
cultivating the cell culture according to claim 9 in vitro; and
recovering the proteins, fish oils or fatty acids.

20. A method of producing proteins comprising enzymes with an optimum activity at low temperatures, fish oils or fatty acids comprising:
cultivating the cells according to claim 6 in vitro; and
recovering the proteins, fish oils or fatty acids.

21. A method of breeding fish comprising reproductive cloning of poikilothermic vertebrates obtained from the cells according to claim 6.

22. A method of implanting foreign gene material or foreign matter comprising introducing the foreign gene material or foreign matter into the cells according to claim 6.

23. A kit comprising the cells according to claim 6, and expedients and/or reagents.

24. The method of claim 18, wherein the chemicals are pharmaceutical drugs and the testing is screening.
